(19) United States
(12) Patent Application Publication
(10) Pub. No.: US 2019/0065854 A1
(43) Pub. Date: Feb. 28, 2019

(54) SYSTEM AND METHOD FOR VISIO-TACTILE SENSING

(71) Applicants: Neel Krishnan, Brooklyn, NY (US); Gina Tibbott, Philadelphia, PA (US); Mitalee Bharadwaj, Kendall Park, NJ (US)

(72) Inventors: Neel Krishnan, Brooklyn, NY (US); Gina Tibbott, Philadelphia, PA (US); Mitalee Bharadwaj, Kendall Park, NJ (US)

(21) Appl. No.: 16/081,764

(22) PCT Filed: Jan. 24, 2017
(86) PCT No.: PCT/US17/14675

§ 371 Date: Aug. 31, 2018

Related U.S. Application Data
(60) Provisional application No. 62/302,872, filed on Mar. 3, 2016.

(51) Int. Cl. G06K 9/00; A61F 9/08; G06F 3/01

(52) U.S. Cl. CPC G06K 9/00671; A61F 9/08; G06F 3/016; G06F 3/014; G06F 3/012

(57) ABSTRACT

Abstract: A device for providing tactile feedback of an environment using a haptic device to allow the physical sensing of objects in an environment. The device uses a plurality of vibrational elements laid out in a grid pattern on a portion of the user's body, and activates various subsets of the vibrational elements in response to the shape of objects in proximity to the user. The strength of the vibration represents distance to the object or various portions of an object.

### RELATED APPLICATIONS

This application claims the benefit of U.S. Provisional Application Ser. No. 62/302,872, filed Mar. 3, 2016

### BACKGROUND OF THE INVENTION

There is a need for devices for assisting visually impaired people in navigating the physical environment, as well as for obstacle identification, discrimination, and avoidance in other scenarios in which vision is compromised and/or additional sensing of the visual environment is required. Examples include firefighting scenarios, nighttime search and rescue in cases where light sources are scarce, and military applications in which night vision goggles are problematic, such as nighttime urban combat. Such devices would also be useful for extending a normally sighted human's capacity to sense the visual environment to areas not in his/her visual field, and for augmenting audio-visual presentations with tactile sensations.

The primary need, however, is for a device which takes recognizes that visually impaired individuals often have heightened abilities to process and comprehend sense information from other senses. The success of Braille suggests that the visually impaired could process tactile information rapidly, and that tactile information may be the best way to augment their ability to physically navigate the world without the use of sight.

### SUMMARY OF THE INVENTION

The device comprises two digital cameras or other visual light or infrared input devices or sensors arranged next to or near each other and with parallel or roughly parallel lines of sight, as shown in FIG. 2a. The input of both cameras is converted to sets of pixel values by a computer chip or chips or by the digital camera itself, and these values are fed into a stereo depth algorithm that transforms them into a visual depth map of the visual environment (the camera and chip are referred to collectively herein as the “input device”). This input device is attached to a set or sets of vibration elements inlaid in a uniform pattern in or on a piece or pieces of fabric or other flexible material and worn on parts of the user's body, for example wrapped around the forearms. FIGS. 1a and 1b show two possible layouts. The visual depth map produced by the stereo depth algorithm is used to create a tactile depth map of the environment across the vibration motors (also referred to as ‘vibration elements’ herein), in which distance between objects in the visual field is represented by the distance between vibrating motors, and the depths, i.e. the distance to the objects from the user, are represented by the intensity or speed (frequency) of the vibration. FIGS. 2a-d show a representation of the device mechanism, described in depth below. The device thereby allows the visually impaired or other user to ‘feel’ the contours and depths of the visual environment around them, and to examine the 3-dimensional layout of specific objects in depth.

The device may also include other sensors, such as, but not limited to, SONAR based sensors, attached to the housing of the cameras or the vibration elements or located elsewhere, which provide additional information about the environment by translating the sensor outputs into other vibrational patterns across the vibrating motors or to other vibration motors worn on other parts of the body. SONAR may be particularly suited to locating the walls of indoor environments, and this information can be augmented by the information produced by the binocular depth algorithm described below for a fuller tactile ‘picture’ of the user's environment.

The device may include hand-held or other toggling devices that allow the user to focus the device on specific objects in the visual field or on a specific subset of the wider visual environment in order to examine them more closely via the tactile output device. For example, in one embodiment accelerometers worn on a headset could allow the user to direct the device to apply the visual-to-tactile discrimination algorithm described below to a specific object or region of the visual environment via head movements in conjunction with input from a hand-held toggling device. These regions of interest could be identified by a standard blob detection or object centroid detection algorithm applied to one or both of the digital cameras' outputs.

### BRIEF DESCRIPTION OF THE DRAWINGS

FIGS. 1a and 1b show two possible layouts of the vibrational elements on the body of a user.

FIG. 2a shows a schematic view of the layout of the cameras in the device.

FIG. 2b shows the representation of the depth map as a heat map, with various colors representing range to the surface of an object.

FIG. 2c shows the heat map of FIG. 2b in aggregated form.

FIG. 2d shows how a user would perceive the object, wherein color values represent varying intensities of vibrations in the vibration elements on the sleeve.

### DETAILED DESCRIPTION

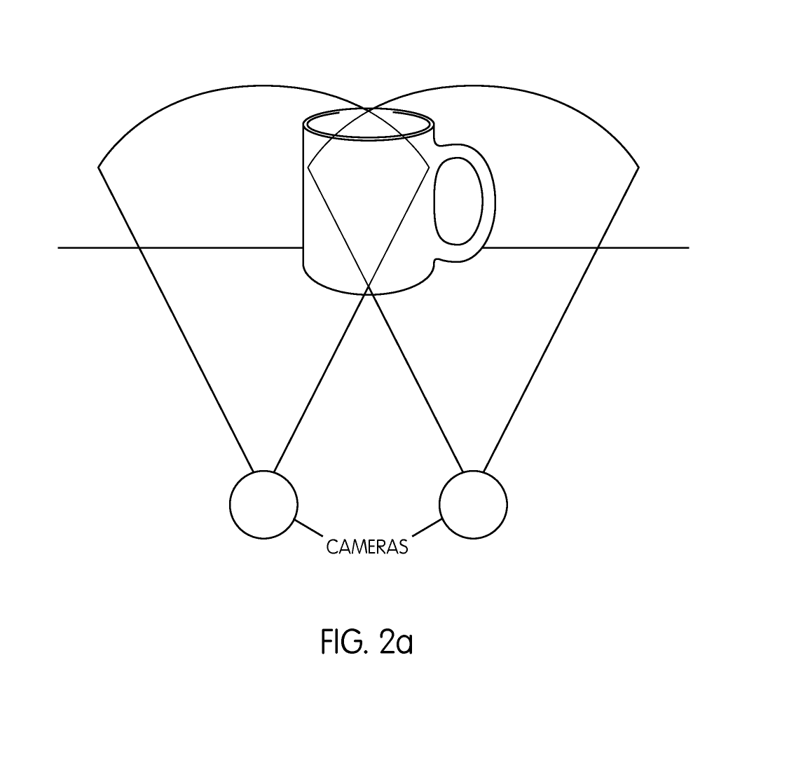

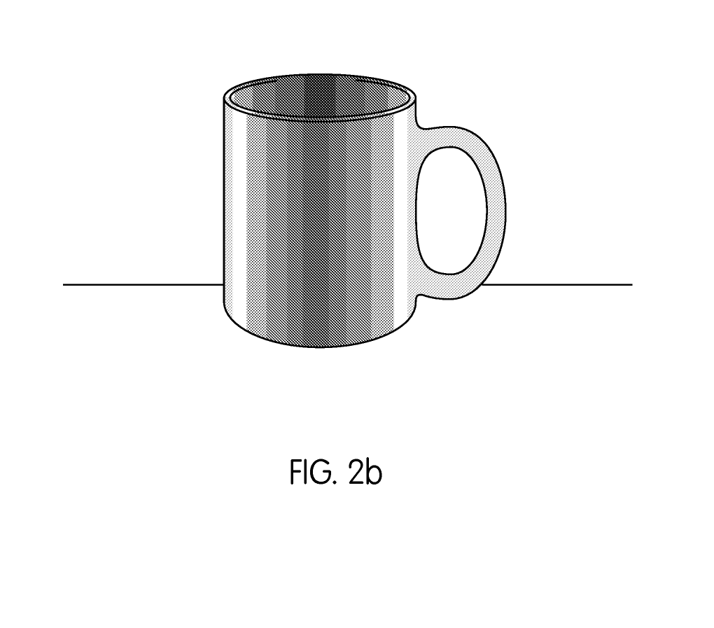

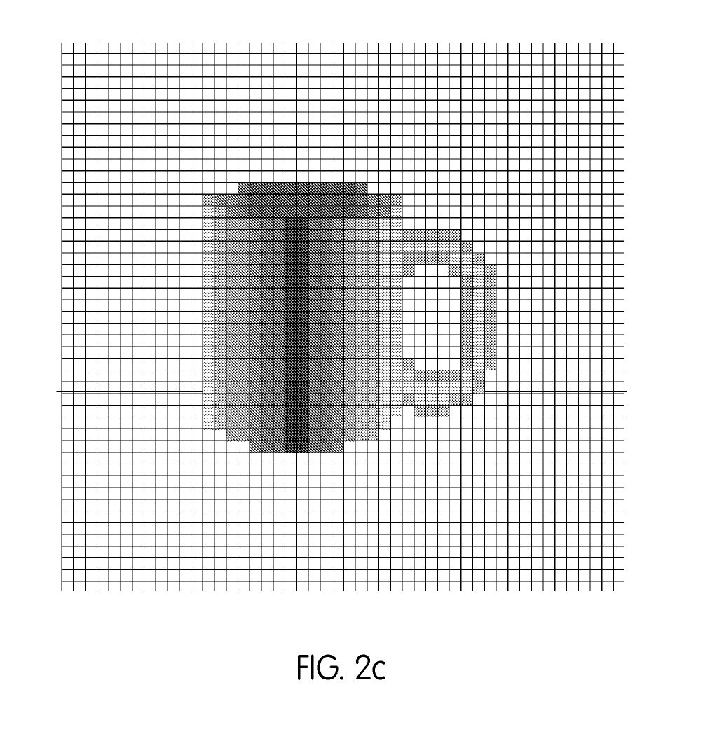

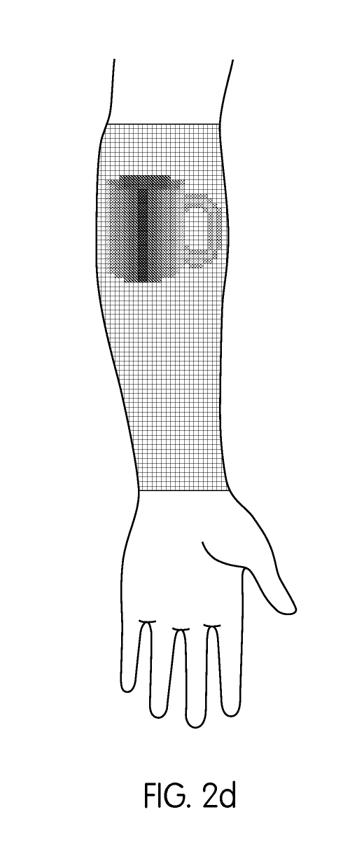

The device comprises a housing containing two cameras arranged with parallel or roughly parallel lines of sight (also referred to herein as the ‘binocular input device’, shown in FIG. 2a). The housing may be mounted on eyeglasses or worn on the head of the user, or attached to other parts of the user's body such as the hand. The two cameras can also be held in independent housings and given parallel or roughly parallel lines of sight by the user or by means of a mechanism connecting the housings. In one embodiment, the camera housings may be equipped with motors to allow the cameras to move independently of each other.

The cameras are in communication with a computer chip or processor which executes software for controlling the system, including any of a number of established stereo depth algorithms used to translate input from binocular digital visual input into a depth map of the observed environment, as shown in FIGS. 2a-b. Examples of such stereo depth algorithms include sum of squared difference (SSD) algorithms that perform pixel matching between the pixels of the two camera images by iteratively finding subsets of pixels in one image that minimize the sum of squared differences between that subset of pixels in a corresponding set in the other image. Other examples augment the SSD algorithm with additional steps that minimize the computation time of the SSD algorithm, for example, adding blob, contour and object centroid detection to determine the major points of interest in the visual environment and to reduce the matching problem to these areas. Other approaches first transform the input images before applying an SSD algorithm or related pixel matching algorithm, for example, by first rotating objects or performing transformation of the colors in or texture of the image. The resulting matched pixel information (i.e. the relative locations in the two camera input fields of the matched subsets of pixels), can be used in conjunction with the distance between the camera lenses to determine the distance to the objects in the visual field from the user by triangulation. This disclosure is not limited with respect to the choice of stereo depth algorithms, many of which are established and well known in the computer vision literature.

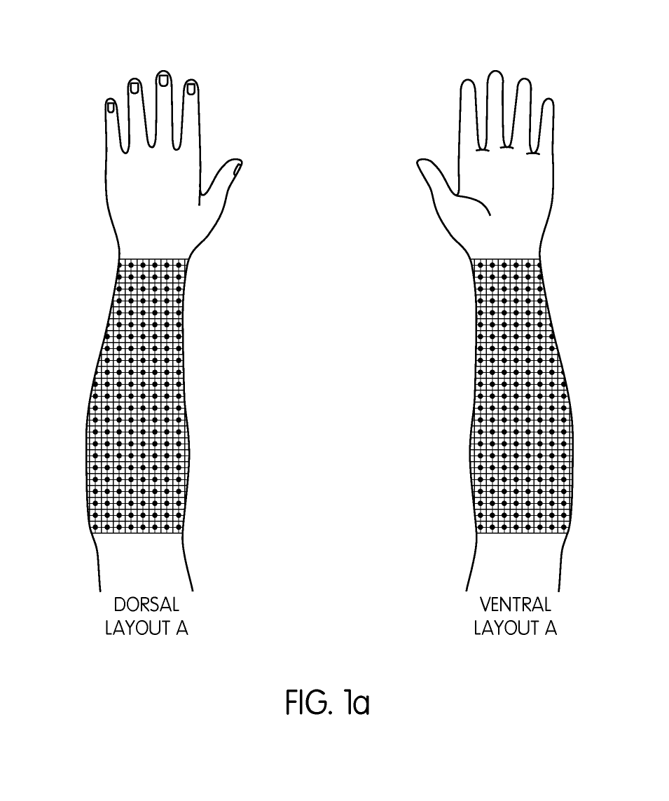

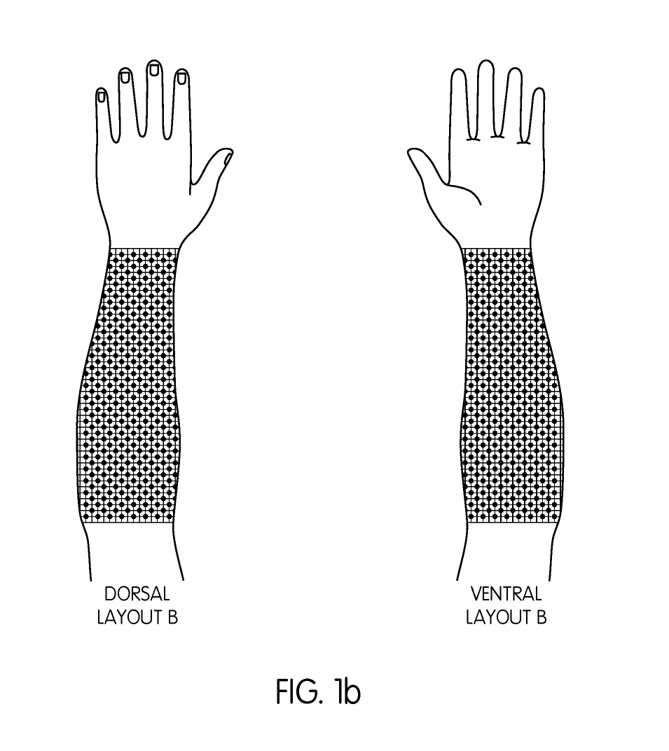

The depth map produced by the above device is used to activate a set of vibrating elements under control of software executing on the processor. In the preferred embodiment, these vibrating elements may be, for example, the type of miniature vibrating motors used in cell phones. These vibrating elements are preferably laid out in a uniform pattern, for example in a grid pattern. FIGS. 1a and 1b show two possible layouts for the vibrating elements. Preferably, the vibrating elements are inlaid in or on fabric or other flexible material such that, if the fabric is worn on a part of the body, the vibrations may be felt on the skin. The vibrating elements themselves and the electric wiring are kept safe from moisture or disturbance or damage from outside objects, for example, through embedding between layers of Gortex or another suitable fabric, forming a haptic feedback device. The fabric with vibrating elements are preferably worn on parts of the body with high tactile sensitivity, such as the forearms, shoulders, or back or front of the hand, and/or areas of the body not normally used for functional sensing, such as the back, or on areas of the face, e.g. around the eyes or on the cheeks or back of the neck. The forearms may be an ideal area for the user to wear the device due to their high tactile sensitivity, and otherwise low usage for functional sensing or use in manipulation of objects by the user. Accordingly, the remainder of this disclosure will refer to the fabric with inlaid vibrating elements as the ‘sleeves’ of the device.

Description of Visual-to-Tactile Sensing Algorithm

FIGS. 2a-d present an overview of how the device aids in visual sensing of the environment for the visually impaired by creating a relationship between visual information and tactile information. It illustrates the device observing a mug and translating a 3d depth map of the object into a sensible vibration pattern felt by the user through the device's sleeves. The mug may have happened to be within the visual field of the user, or the user may have adjusted the housing of the cameras to train on the mug using head movements or other movements in response to tactile feedback from the device. The illustrations abstract away other details of a probable environment for the mug, such as walls of the room and the table it sits on, though the device can also sense the walls of the user's environment and other details in a similar way. For example, if the full visual field of the cameras included walls of the room, a table and a mug, the acquisition step below would observe all of these features and process them in the subsequent steps executed by the software into a tactile map communicated to the user via the device sleeves. FIGS. 2a-d abstract away these other details for simplicity of presentation.

The device can also be used to train on a specific object occupying a sub-region of the visual field and communicate its depth information to the user in the way shown, for example, a sub-region identified within the image by a standard blob detection or object centroid detection algorithm and chosen by the user via head movements read by accelerometers mounted on the head, or identified using other toggling mechanisms such as hand held devices which communicate with the computer chip which is executing the depth discrimination algorithm to identify regions to focus on within the input images.

The following steps illustrate the full algorithm executed by the device to acquire and translate visual information into tactile information felt by the user.

Step 1: Acquisition

In the acquisition step, shown in FIG. 2a, the mug is observed by the two cameras with parallel or roughly parallel lines of sight, and the digital image information, for example, a matrix of pixel color values (for example, RGB 3-tuples), for each camera, is fed to the stereo depth algorithm contained in software being executed by the processor.

As noted above, in this step, the two cameras could also acquire two pictures of the whole visual environment instead of a specific region or object within it, and perform the following steps on that set of images, also implemented in the software executing on the processor.

Step 2: Depth Discrimination

In the second step, any one of a number of standard stereo depth algorithms are applied to the two pixel matrices to produce a single depth map of the object. This depth map is a matrix of numerical values representing the distance from the user of the corresponding aspect of the visual image of the object. The depth map is represented in FIG. 2b as a heat map wherein colors toward the red end of the spectrum represent parts of the mug that are closer to the user, and colors towards the blue end of the spectrum represent aspects of the mug that are farther away.

Step 3: Aggregation

The matrix of vibrating motor elements on the sleeves, shown in FIGS. 1a-b, which communicate the depth information to the user, will be less granular than the depth map produced in step 2, given current economically viable vibration motor technology. Because a single vibrating element must vibrate with a single level of speed/intensity, to display the depth map produced in step 2 across the vibration elements, the device may apply an aggregation algorithm to average subsets or regions of the depth map into single values, allowing that region to be represented by a single vibrating element. For example, the depth values in a window of 10×10 pixels could be averaged and that value used to activate a single vibrating element that corresponds spatially to that region. The rest of this disclosure will refer to the size of the window of pixels corresponding to each single vibration element as the ‘translation granularity’.

While a number of different aggregation algorithms could be applied, in one embodiment the subsets can be visualized as adjacent tiles of the visual map. The tile pattern can be seen in FIG. 2c as the parts of the depth map underneath each box on the grid pattern. The algorithm averages all of the depth values within each grid tile into a single depth value, for example, by taking the simple average of all the depth values in that region, or taking a weighted average with some weighting scheme, for example, one that places higher weights on the values towards the middle of the tile.

The resulting aggregated matrix of depth values is less granular than the depth map produced in step 2. The result is illustrated in FIG. 2c, in which each box in the grid represents a different depth value, shown as the single colors in each grid box.

Step 4: Tactile Activation

In the final step, the aggregated grid of depth values is communicated to the user as vibrations felt on the device's sleeves. In FIG. 2d, the color values represent varying intensities of vibrations in the vibration elements on the sleeve, with colors towards the red part of the spectrum representing more intense vibrations and colors towards the blue end of the spectrum representing less intense vibrations. Uncolored boxes on the grid represent vibration elements that are not vibrating. Thus the vibrational patterns on the sleeves in FIG. 2d allow the user to feel the depth contours of the mug indexed by the aggregated depth map in FIG. 2c. If another object were also present in the user's visual field, the corresponding vibration pattern would be separated from the mug by a distance proportional to the observed distance between the two objects in the visual field. The vibration elements are in communication with the processor and are activated and deactivated under control of the software executing on the processor.

If the device is not focusing on a particular object in a subset of the user's visual field in response to user toggling, it performs steps 1-3 on the entire visual field. In this mode the visual environment surrounding the mug would also be communicated as vibration patterns in the un-activated motors in FIG. 2d. In one embodiment, a threshold can be set such that, for depth values above this threshold, the vibration element is not activated. This value can be set either as a fixed value or using software that adapts to the visual environment, for example by taking an average depth value for the whole visual field observed by the cameras and only communicating to the sleeves depth values of a magnitude above this average minus some multiple of the standard deviation of the full depth value distribution.

The algorithm in steps 1-4, shown in FIGS. 2a-d, are repeated iteratively several times a second so that the user experiences the output as a substantially real-time haptic representation of the visual environment or of whatever object or sub-region of the surrounding visual environment the user is directing the device to focus on via the toggling device alluded to above, and described further below.

Description of Possible ‘Focus’ Toggling Devices

To allow the user to focus on a specific object or region of the visual environment for tactile sensing, an optional toggling device or devices, for example held in the hand of the user and squeezed between thumb and forefinger, can be used to narrow the visual field examined by the stereo depth algorithm and translate this information to the vibrating sleeves such that individual objects and smaller sub-regions of the visual environment can be examined in closer detail. In this case, the toggling device would narrow the visual field being examined by the stereo depth algorithm, that is, reduce the size of the square or rectangular subset of one camera's input pixel matrix that is matched by the stereo depth algorithm to the image in the other camera to produce the depth map in step 2 above, while simultaneously decreasing the translation granularity value, such that a smaller area of the visual field corresponds to each vibrating element activated. The user can thus focus on a particular object or sub-region of the visual field for tactile sensing.

For example, in one embodiment, objects of interest in the visual environment can be identified by a blob detection or object centroid detection algorithm. To focus on the specific object, the user moves their head such that the object is oriented near the center of what would be their visual field, and then squeezes the hand-held toggling device. In response to the pressure on the toggling device, the device narrows the visual field being processed by the visual-to-tactile algorithm in steps 2-4 to that occupied by the object, and decreases the translation granularity value such that fewer visual pixels correspond to each vibrating motor element, allowing the user to sense finer details of the object or region.

Hardware Implementation

GPU chips are actively used in visual processing operations to achieve orders-of-magnitude improvements over CPUs in visual processing applications. In various embodiments, the visual-to-tactile algorithm described above is executed iteratively several times a second using a GPU chip, a generic microprocessor other computer hardware used for high throughput specialized computing applications, such as a field-programmable gate arrays (FPGA's) or an application-specific integrated circuit (ASIC's), so as to present a close-to-real-time tactile representation of the environment in the device's sleeves.

Usage of the Device

The device achieves real-time tactile representation of the environment by iteratively mapping the stereo depth map to the vibrating motor elements in the sleeves, i.e. steps 1-4 above, rapidly in time such that the distances to objects in the user's current visual field are represented by the intensity of the vibrations, whereas the spatial relationship between the objects in the visual environment correspond to distances between the vibrating elements. The resulting visio-tactile map allows a visually impaired user to feel the visual layout of the environment via haptic sensing, and even to feel the 3-dimensional contours of the objects themselves through discrimination of the relative depths of the different parts of objects. If the binocular input device is worn on the user's head or hands, the user may explore the environment by training the cameras on various parts of the environment and feeling the resulting change in the vibrational map. If the binocular input device is used with infrared cameras or other sensors suited to sensing low light or light outside of the visual frequency range, the device could also be used for sensing depth in darker environments.

Multi Sensor Variation

While stereo depth algorithms produced using binocular inputs from cameras or other visual sensors produce a large amount of useful information for navigating the environment, there are certain objects and scenarios that are known to produce difficulties for these algorithms. For example, walls may frustrate efforts at stereo depth discrimination since their generally uniform visual appearance may render pixel matching between the two visual input devices difficult. Another example is occlusions: objects that appear in one camera may not appear in the other due to visual obstructions or a transient lack of roughly parallel orientation between the two visual input devices.

To deal with these and other problems, in several embodiments the device presented above may be used in conjunction with technology found in other forms of visually-impaired navigation aides. For example, SONAR sensors or infrared rangefinders worn on the hands or on the device's sleeves or elsewhere could be used to augment the visio-tactile map by sensing the distance to walls or other larger obstacles and presenting them as vibrations that vary in intensity according to the distance to the obstacle. For ease of interpretation by the user, the readings from these ancillary sensor devices could be presented as vibrations on elements placed on other parts of the body, such as the back of the hand, the upper arm or shoulder, or the back, or in any case separate from the output device which presents the information produced by the stereo depth algorithm described above. The readings could also be integrated into the output of the primary vibration sleeves via various computational strategies.

Moving Cameras Variation

In another variation of the device presented above, the visual input devices or cameras could be mounted on rotating housings and moved by servos wired into the computer chip. Stereo depth matching could then also be achieved by moving one or both cameras so as to produce a correspondence between a subset of pixels in the input of one visual input device and that of the other visual input device, as measured by an SSD or similar algorithm. The relative rotational angles of the cameras could then be used in conjunction with the distance between the camera lenses to determine the distance to the object or objects. Such a mechanism would imitate the focusing and depth discrimination of the human eye via tensing of the ocular muscles.

Other Extensions

The availability of cameras on the device means it could also integrate other useful extensions for the visually impaired. For example text on signs and embedded in other areas of the visual environment could be identified and read by extant computer vision algorithms, and this text could either be read audibly to the user via headphones or a speaker or output on a Braille reading device.

We claim:

1. A visio-tactile sensing system comprising:
a. one or more input devices for providing one or more images of an environment;
b. a haptic feedback device; and
c. a processor programmed to:
i. extract range and shape information of objects in said environment from said one or more images; and
ii. control said haptic feedback device to provide a user with non-visual range and shape information regarding said objects.

2. The system of claim 1 wherein said one or more input devices comprise two digital cameras arranged in a stereo configuration having approximate parallel lines of sight.

3. The system of claim 2 further comprising a housing for disposition on the head of a user of said system for holding said cameras in said stereo configuration.

4. The system of claim 2 wherein said processor is executing a stereo depth algorithm to create a depth map from said one or more images, said depth map representing said range and shape information of said objects.

5. The system of claim 4 wherein said haptic feedback device comprises:
a. a flexible material suitable for placement on the body of a user of said system; and
b. a plurality of vibrational elements held in a grid configuration by said flexible material.

6. The system of claim 5 wherein said processor controls said vibrational elements to create a tactile depth map using said vibrational elements.

7. The system of claim 6 wherein said tactile depth map:
a. represents the shape information of said objects by activating a subset of said vibrational elements representing a two-dimensional shape of said objects; and
b. represents range information of said objects by varying the speed or intensity of said subset of said vibrational elements.

8. The system of claim 7 further comprising a focusing function which allows a user of said system to focus said system on a single object in said environment.

9. The system of claim 8 wherein said focusing function comprises a blob detection algorithm for detecting objects in the field of view of said cameras; and wherein a user may select a detected object upon which said system will focus.

10. The system of claim 9 wherein said object is selected using a toggling device held in the hand of a user of said system.

11. The system of claim 10 wherein said object is selected when the user places the object in the center of the field of view of said cameras.

12. The system of claim 1 further comprising:
a. a secondary input device for providing auxiliary information about said environment; and
b. a secondary haptic device for providing a user with non-visual information regarding said environment.

13. The system of claim 12 wherein said secondary input device is a SONAR.

14. A haptic feedback device comprising:
a. a flexible material shaped to be worn on a part of the body of a user of said system; and
b. a plurality of vibrational elements arranged in a grid pattern and mounted on or in said flexible material.

15. The haptic feedback device of claim 14 wherein a subset of said vibrational elements may be activated.

16. The haptic feedback device of claim 15 wherein the speed or intensity of the vibration of said subset of vibrational elements may be varied.

17. A method of providing haptic feedback of objects in an environment comprising the steps of:
a. acquiring image information of an object within said environment from two cameras arranged with approximate parallel lines of sight;
b. creating a depth map based on said image information, said depth map comprising a matrix of numerical values representing the distance between said cameras and the surface of said object;
c. applying an aggregation algorithm to average subsets or regions of said depth map into single values; and
d. activating one of a plurality of vibrational elements arranged in a grid pattern for each of said single values.

18. The method of claim 17 wherein single values said depth map have a spatial correspondence with a single vibrational element within said grid of elements.

19. The method of claim 18 wherein each vibrational element is activated using a varying speed or intensity based on its corresponding single value in said depth map.

20. The method of claim 19 wherein said plurality of vibrational elements is held in said grid pattern by mounting on or in a flexible material shaped to be to be worn on a portion of a human body.
